(19) United States
(12) Patent Application Publication
(10) Pub. No.: US 2006/0222692 A1
(43) Pub. Date: Oct. 5, 2006

(54) Method and compositions for transdermal administration of antimicrobial medications

(75) Inventor: Edward M. Lane, Weston, CT (US)

Correspondence Address: ROTHWELL, FIGG, ERNST & MANBECK, P.C., 1425 K STREET, N.W., SUITE 800, WASHINGTON, DC, 20005, US

(73) Assignee: Fairfield Clinical Trials LLC, Bridgeport, CT (US)

(21) Appl. No.: 11/094,511

(22) Filed: Mar. 31, 2005

(51) Int. Cl. A61K 38/14; A61K 31/7048; A61K 31/7034; A61K 31/545; A61K 9/70

(52) U.S. Cl. 424/449; 514/28; 514/29; 514/37; 514/8; 514/192; 514/200

(57) ABSTRACT

The present invention relates to methods and compositions for improved efficacy and delivery of time-dependent antimicrobial drug compositions to a patient. Transdermal dosage forms and methods for steady-state delivery of drug to produce and maintain a serum concentration of drug above the minimum inhibitory concentration or minimum microbicidal concentration are provided.

### BACKGROUND OF THE INVENTION

1. Field of the Invention

The present invention relates generally to medical compositions and methods for administration of antibacterial, antiviral, antifungal, antimycobacterial, antihelmintic and antiprotozoan pharmaceutical compositions. In particular, this invention relates to methods and compositions for transdermal administration of time-dependent antimicrobial drugs which combat organisms of human pathology.

2. Description of the Background Art

Antimicrobial compositions to treat infectious diseases are known and have been in use for centuries. These drugs have long-recognized clinical benefits in treating infectious disease and parasitic or protozoan diseases. As used herein, the terms microbe, microbial, antimicrobial, microbistatic and microbicidal all refer to any organism such as bacteria, mycobacteria, viruses, fungi, amoebae, protozoa, helminths and the like which cause or can cause human pathology, infection or infestation, and includes single-celled or multicellular organisms. Antimicrobial drugs often are classified according to their activities against various organisms; compounds useful to combat each of these types of organisms are known in the art.

The pharmacokinetics and pharmacodynamics of drugs are known to influence their effectiveness against microbes. Generally, to interpret how a drug behaves in the body, the serum or plasma concentration of the drug is measured over time and plotted on a graph. The peak concentration achieved after a dose of drug is termed the Cmax. This pharmacokinetic parameter indicates the maximum serum concentration of drug which is attained. The area under the concentration/time curve (AUC) indicates the amount of drug which is bioavailable and which can exert an effect systemically. Administration of drugs in periodic dosages, as is usual with any orally administered drug, results in sequential peaks and troughs in the drug concentration. For antimicrobial drugs, the minimum inhibitory concentration (MIC) or minimum microbicidal concentration (MMC) is the concentration of drug required to inhibit growth of or kill the microbe in vitro. The MIC or MMC should be reached in serum during treatment, and preferably maintained, in the serum for the antimicrobial drug to be effective.

An important distinction among antimicrobial drugs is whether the antimicrobial effect of the drug is concentration-dependent or time-dependent. For concentration-dependent drugs, favorable resolution of the infection is related to the peak serum concentration (Cmax) of the drug at the site of the infection. These drugs exhibit a “post-antibiotic effect” (also termed “prolonged” or “persistent” effect) and inhibit multiplication of the organism for a prolonged time even after the drug concentration wanes, provided that the concentration at the site of the infection has reached the MIC or MMC. Greatest benefit of these drugs is achieved by ensuring that the peak concentration (Cmax) is maximized and in no case fails to reach the MIC or MMC at the site of infection. The presence of significant troughs in serum drug concentration are not of concern in most cases.

Time-dependent drugs (also sometimes referred to as “non-concentration-dependent” or “concentration-independent” drugs), on the other hand, have a less noticeable post-antibiotic effect in most cases and require the concentration of drug to be maintained at or above the MIC or MMC consistently during the entire course of treatment to be most effective. For drugs of this type, the most effective killing is achieved when the drug is above the MIC or MMC for at least 80% and preferably more of the time after dosing. Time-dependent drugs, in general, do not exhibit dose-responsive killing above the MIC, so higher concentrations above the MIC or MMC, and particularly above four times the MIC or MMC, do not produce significant additional benefit. Their activity generally is not rapid. To be most effective, time-dependent antimicrobial drugs should be administered in a consistent manner, without allowing concentration of the drug to drop below the MIC or MMC.

Time-dependent antimicrobial drugs, as the term is used here, are microbistatic or microbicidal compositions for which the inhibitory or killing rate of the composition is not dependent or is minimally dependent on the concentration of the composition at the site of the infection above the MIC or MMC and which have a minimal or only moderate post antibiotic effect. Examples of clinically important time-dependent antimicrobial compositions include Penicillins, Macrolides, Ketolides, Cephalosporins and Streptomycin.

Maintaining the serum concentration at or above the MIC for extended periods of time, and preferably for the entire course of antibiotic therapy, is highly desirable, and even essential to achieving maximum benefit from a time-dependent antimicrobial drug. Traditional oral dosing of time-dependent antimicrobial drugs often leads to undesirably low minima in serum drug concentration between doses, which can compromise the success of the therapy. To avoid these minima, it may be necessary either to substitute a concentration(dose)-dependent drug or to deliver the time-dependent drug by constant intravenous infusion or more frequent oral dosing. Intravenous infusion of drug, while effective in many cases, has a number of disadvantages such as inconvenience, expense, the need for hospitalization or trained care during administration, and the possibility of infection at the injection site. Frequent oral dosing reduces patient compliance, which may lead to increases in the time that the serum concentration falls below the MIC, which in turn may lead to the emergence of antimicrobial resistance. Therefore, it would be highly desirable to be able to administer these drugs such that the minima associated with traditional dosing did not occur, with serum concentration of the drug maintained at or above the minimum inhibitory concentration, without having to resort to intravenous infusion over long periods.

### SUMMARY OF THE INVENTION

Accordingly, a general object of the present invention is to provide a method and device for administration of time-dependent antimicrobial drugs transdermally.

The invention provides, in one embodiment, a method of improving the efficacy of and reducing the emergence of resistance in a time-dependent antimicrobial drug which comprises administering the antimicrobial drug transdermally. The antimicrobial drug may be an antibacterial drug, an antimycobacterial drug, an antiviral drug, an antifungal drug, and antiprotozoan drug, an anthelmintic drug or any drug effective against a microbe as the term is used herein. Preferably, the antimicrobial drug is selected from the group consisting of penicillin, amoxicillin, oxacillin, dicloxoacilline, clavulinic acid with a penicillin, bicillin, ticarcillin, piperacillin, taxobactam, cephalexin, cefazolin, cephaclor, ceftibuten, cefuroxime, cefprozil, cefotaxime, ceftazidime, cefepime, cifdinir, ceftriaxone, cefditoren, cefpodoxime, aztreonam, ertapenem, cefoxitin, meropenem, imipenem, erythromycin, clarithromycin, azithromycin, telithromycin, clindamycin, daptomycin, cycloserine, quinupristin, dalfopristin, streptomycin, vancomycin, linezolid and combinations thereof.

Another embodiment of the invention provides a method of treating a microbial infection in a patient with decreased risk of the emergence of resistance which comprises administering a time-dependent antimicrobial drug transdermally to said patient. The time-dependent antimicrobial drug may be an antibacterial drug, an antimycobacterial drug, an antiviral drug, an antifungal drug, and antiprotozoan drug, an anthelmintic drug or any drug effective against a microbe as the term is used herein. Preferably, drugs for use with the invention are selected from penicillin, amoxicillin, oxacillin, dicloxoacilline, clavulinic acid with a penicillin, bicillin, ticarcillin, piperacillin, taxobactam, cephalexin, cefazolin, cephaclor, ceftibuten, cefuroxime, cefprozil, cefotaxime, ceftazidime, cefepime, cifdinir, ceftriaxone, cefditoren, cefpodoxime, aztreonam, ertapenem, cefoxitin, meropenem, imipenem, erythromycin, clarithromycin, azithromycin, telithromycin, clindamycin, daptomycin, cycloserine, quinupristin, dalfopristin, streptomycin, vancomycin, linezolid, albendazole and mebendazole.

A preferred embodiment of the invention provides a transdermal drug delivery device comprising a time-dependent antimicrobial drug and a pharmaceutically acceptable excipient. Most preferably, the time-dependent antimicrobial drug may be an antibacterial drug, an antimycobacterial drug, an antiviral drug, an antifungal drug, and antiprotozoan drug, an anthelmintic drug or any drug effective against a microbe as the term is used herein.

Suitable drugs for use in the invention include, but are not limited to penicillin, amoxicillin, oxacillin, dicloxoacilline, clavulinic acid, bicillin, ticarcillin, piperacillin, taxobactam, cephalexin, cefazolin, cephaclor, ceftibuten, cefuroxime, cefprozil, cefotaxime, ceftazidime, cefepime, cifdinir, ceftriaxone, cefditoren, cefpodoxime, aztreonam, ertadenem, cefoxitin, meropenem, imipenem, erythromycin, clarithromycin, azithromycin, telithromycin, clindamycin, daptomycin, cycloserine, quinupristin, dalfopristin, streptomycin, vancomycin and linezolid. The transdermal devices of the invention may include antimicrobial cocktails comprising more than one time-dependent antimicrobial drug.

Additional embodiments of the invention provide a method of treating a microbial infection in a patient which comprises administering to said patient a transdermal drug delivery device as described above. Further, certain embodiments of the invention include a method of systemically providing an antibacterial treatment to a patient while avoiding gastrointestinal side effects of said treatment, which comprises administering to said patient and a method of systemically providing an antibacterial drug to a patient while lessening the likelihood of emergence of drug resistance to said drug, which comprises administering to said patient a transdermal drug delivery device such as those described above.

### DETAILED DESCRIPTION OF THE PREFERRED EMBODIMENTS

In summary, this invention relates to methods and devices for transdermal delivery of time-dependent antimicrobial compounds. Transdermal delivery of drugs has benefits which overcome the disadvantages of current antimicrobial medications described above by delivering a steady-state of drug compound to the patient (zero order kinetics). The dosage is steadily applied to the patient over long periods, usually one to seven days, without either the pain and inconvenience of multiple intramuscular or intravenous dosing or the pain, inconvenience and risk of an indwelling intravenous catheter (such as a central line or a peripheral intravenous longline catheter (PIC) line). The result of the inventive methods is an increase in the effectiveness of the time-dependent antimicrobial drugs which has several additional benefits not present in currently used antimicrobial therapy. For example, the necessary duration of the course of therapy is reduced while chances of the development of resistant microbes also is reduced.

Transdermal delivery of antimicrobials provides zero-order pharmacokinetics, which provides a controlled dose to the patient at the desired level without the peaks and troughs associated with periodic dosing. This form of delivery can be tailored to maintain serum levels above the MIC or MMC, or about 2-4 times the MIC or MMC, but in a sustained manner. This provides the benefits of constant intravenous infusion without the inconvenience and safety concerns associated with intramuscular injection dosing or with an intravenous line and/or pump. For microbicidal drugs with time-dependent killing, this method of administration increases effectiveness of the drug; for microbistatic drugs with time-dependent inhibition, this method of administration increases effectiveness, but also reduces the likelihood of the emergence of resistant strains because the therapy is effectively inhibiting microbial growth at all times during therapy without breaks in effectiveness that allow resistant strains to emerge. Therefore, certain antimicrobial drugs thought to be useless because of resistance emergence may be delivered according to the invention.

An additional benefit of transdermal administration of these drugs is that infections are cleared with shorter courses of therapy when the MIC or MMC is exceeded at least 80% or 90% of the time and preferably 100% of the time during the course of therapy. Short courses of therapy in which the drug serum concentration falls below the MIC or MMC are the most likely scenario for formation of resistance. For this reason, longer courses must be given to each patient, particularly when the serum concentration may drop below the MIC or MMC during therapy. Using the methods of this invention, however, these longer therapeutic regimens are not necessary to reduce resistance because the MIC or MMC easily can be exceeded 100% of the time.

A further benefit of consistent administration of the drug over time to maintain a constant serum drug concentration is the lessening of certain dose-dependent side effects, for example the electrocardiographic QTc interval prolongation seen with Macrolide antibiotics. When high Cmax concentrations, such as are inescapable with oral dosing, are avoided with the present invention, a high steady-state serum concentration can still be provided to the patient without higher peaks in concentration that can lead to the side effect. In addition, when antibiotics are given orally, gastrointestinal symptoms are common due to damage to the natural flora of the gut. Vaginal candidiasis, as well as vomiting, diarrhea and other gastrointestinal symptoms are frequently seen, including Clostridium difficile colitis. These types of side effects can be greatly reduced when the antimicrobial is administered according to the invention. First pass metabolism through the liver also is avoided with transdermal administration.

With transdermal delivery, drugs that cannot be administered orally and now are administered intramuscularly or intravenously, such as vancomycin and certain cephalosporins (e.g., rocephin and cefoxitin) may be administered according to the invention with greater convenience. Oral medications, even if beneficial when dosed orally, often have a very unpleasant taste, which can result in compliance problems, particularly for young children. Drugs that are not formulated for pediatric use due to intractable taste may be used for children when administered according to the invention. Patient compliance is about 90% for once-a-day-dosing, about 80% for twice-a-day dosing and about 50% for three times-a-day dosing. The methods of this invention allow drugs that formerly required frequent dosing when given orally for maximum effectiveness to be dosed once-a-day or less frequently, since transdermal patches may be applied up to only once per week. Transdermal delivery according to the invention therefore increases patient compliance.

Most importantly, time-dependent antimicrobial drugs are more effective when the serum concentration is controlled. Transdermal delivery of time-dependent antimicrobials maintains the serum concentration above the MIC or MMC for the entire course of therapy, allowing clearance of the infection in less time with less drug administered and fewer side effects. The goal of therapy with this class of drug according to the invention is to quickly achieve a serum concentration above the MIC or MMC and to maintain the serum concentration above the MIC or MMC for 100% of the remainder of the course of treatment. Preferably, the serum concentration should be maintained above the MIC or MMC for at least 80% of the course of treatment and most preferably for at least 90% or at least 99% of the time. Doses of about two to four times the MIC or MMC also may be used. MIC data is determined by the consensus acceptance of standards established by the National Committee for Clinical Laboratory Standards (NCLLS) and the Clinical and Laboratory Standards Institute (CSLI), in vitro and is therefore available to those of skill in the art. In vitro sensitivity is accepted at less than 8 μg/mL for aerobic organisms and less than 16 μg/mL for anaerobic bacteria. Therefore, persons of skill in the art can easily determine a suitable dose for antimicrobial drugs to achieve a serum concentration that is appropriate, based on this information.

Dosages and desirable steady-state plasma or serum concentrations of antimicrobial drugs can be determined by any skilled physician or other person of skill in the art. The minimum inhibitory (or microbicidal) concentrations of these drugs are known in the art, therefore those of skill in the art have some experience in determining and effective and preferred dosages which exceed the effective dose of a time-dependent antimicrobial compound. There is considerable experience in the prior art in formulating oral or intravenous dosage forms of many of these antimicrobial compounds, for example. Therefore, useful serum concentrations for practice of this invention can easily be determined by the person of skill, keeping in mind that dosages should be maintained to produce serum or plasma concentrations above the MIC or MMC of the drug in question. Doses of up to twice, three times or four times the MIC or MMC of the drug may be used, provided that concentrations are low enough to avoid toxicity. The exemplary serum concentrations in Table II for exemplary drugs are given to provide guidance for practice of the invention, but are not intended to be limiting. MIC/MMC data for particular organisms are available in the art and/or can be determined in vitro. See Table II, below. Duration of treatment can be determined by the person of skill as a matter of routine. In general, treatment for common infections may be continued for 1-14 days, preferably 3-10 days or 5-10 days, for example 5 days, 7 days, 10 days, or 14 days. For some infections, however, it is known that much longer treatment is necessary, for example 30 days, or even up to one year.

Transdermal delivery systems suitable for systemic administration of known drug compositions are suitable for the methods of the invention. A system for administration of time-dependent antimicrobial drugs generally comprises a backing layer, at least one reservoir containing the active substance and an adhesive for attachment to the skin of the user.

The phrase “transdermal drug delivery device” or “transdermal device” refers to any dosage form suitable for systemic administration of a pharmaceutical compound through the skin. Preferred transdermal drug delivery devices are commonly known as patches. Examples of transdermal devices include any of the known types of transdermal patches such as drug-in-adhesive, matrix and reservoir transdermal patches and can include any preparation designed for transdermal delivery of an active pharmaceutical compound, such as ointments, liposomal and microsomal lotions or emulsions, adhesive films and the like. As used in this application, the term “patch” is intended to be interchangeable with the phrase “transdermal drug delivery device” and encompasses all dosage forms for systemic, transdermal delivery of a drug.

Transdermal drug delivery devices for use with the invention can be of any design known in the art, including specialized patches for iontophoretic delivery or in conjunction with small electric currents (electroporation), ultrasound or microneedle technology to assist delivery across the skin. Suitable patches may include any type of transdermal device technology known to the art, with or without a rate-limiting membrane to control diffusion of the active ingredient(s) to the skin. Transdermal drug delivery devices can be constructed with a reservoir, matrix or adhesive which contains the drug for delivery to the skin of a patient. Exemplary suitable transdermal technologies which are compatible with the present invention include those used in, for example, D-TRANS™, E-TRANS™, MICROFLUX™, LATITUDE™, LATITUDE™ DUO, CLIMARA PRO™, for example. Any known type of transdermal delivery device or system may be used with the embodiments of this invention.

In drug-in-adhesive patches, a drug is dissolved or suspended directly in the adhesive which contacts the skin. Reservoir transdermal systems include a liquid or semi-liquid compartment containing a drug suspension or solution, separated from the skin by a semi-permeable membrane. In matrix transdermal systems, a drug is contained within a solid or semi-solid matrix which contacts the skin of the user and is surrounded at the perimeter by an adhesive. These different transdermal systems are described in, for example, U.S. Pat. Nos. 4,751,087; 5,372,819; 5,405,317; 6,312,715; 6,322,532, the disclosures of which are hereby incorporated by reference.

Table I. Exemplary Time-Dependent Antimicrobial Drugs.

A. Antibacterial Drugs

Penicillins, e.g., benzylpenicillin, amoxicillin, ticarcillin, piperacillin; Cephalosporins, e.g., cefpodoxime, cefuroxime, cefazolin, cefalor, ceftibuten, cefprozil, cefotaxime, ceftazidime, dephaloexin, cefepime, cefdinir, ceftriaxone, cefditoren; Macrolides, e.g., erythromycin, clarithromycin, spiramycin, roxithromycin, azithromycin; Carbapenems, e.g., imipenen, meropenem; β-lactams, e.g., meropenem, Monobactams (e.g., aztreonam), ertapenem, cefoxitin, imipenem; Ketolides, e.g., telithromycin; Glycopeptides, e.g., vancomycin; Lincosamides, e.g., clindamycin, lincomycin; Cyclic lipopeptide antibacterial agents, e.g., daptomycin; Streptogamins, e.g., quinupristin, dalfopristin; Tetracyclines, e.g., doxycycline, minocycline, tigecycline; Diarylquinoline; Oxazolidinones, e.g., linezolid.

B. Antimycobacterial Drugs

Rifampin, Rifabutin, Cycloserine, Isoniazid, Ethambutol, Pyrazinamide.

C. Antiviral Drugs

Cidefovir, Foscarnet, Ganciclovir, Valganciclovir, Formivirisen, Zidovudine, Zalcitabine, Didanosine, Stavudine, Lanivudine, Tenovir, Emtricitabine, Nevirapine.

D. Antifungal Drugs

Fluconazole, Voriconazole, Itraconazole, Caspofungin, Clotrimazole, Amphotericin B, Micafungin, Terbinafine, Naftifine, Natamycin, Butenafine, Amorolfine, Ravuconazole, Posaconazole, Flucytosine, Econazole, Enilaconazole, Miconazole, Oxiconazole, Saperconazole, Sulconazole, Terconazole, Tioconazole, Nikkomycin Z, Anidulafungin (LY303366), Nystatin, Pimaricin, Griseofulvin, Ciclopirox, Haloprogin, Tolnaftate, Undecylenate.

E. Anthelmintic Drugs

Mebendazole, Niclosamide, Praziquantel, Pyrantel, Thiabendazole, Albendazole, diethyl carbamazine, Ivermectin, Benzimidazole, Praziquantal/Benzimidazole combination.

F. Antiprotozoan Drugs

Pyrimethamine, Sulfadiazine, Clindamycin, Mebendazole, Thiabendazole, Chloroquine.

TABLE II
Exemplary Serum Concentrations of Time-
Dependent Antimicrobial Drugs.

| Drug | Approximate MIC^{a} (μg/mL) |
|---|---|
| penicillins | 2 |
| cephalosporins | 4 |
| macrolides | 1 |
| ketolides | 1 |
| Meropenem | 1 |
| Imipenem | 1 |
| Clindamycin | 0.5 |
| Tetracycline | 3.6 |
| Rifampin | 0.5 |
| Doxycycline | 0.03 |
| Chloramphenicol | 4 |
| Daptomycin | 0.5 |
| Cycloserine | 32 |
| Quinupristin/ | 8 (combination) |
| Dalfopristin | (enterococcus) |
| Quinupristin/ | 1 (combination) |
| Dalfopristin | (Staph. aureus) |
| Streptomycin | 2 |
| Vancomycin | 16 |
| Linezolid | 2 |
| Pyrimethamine | 1 (toxoplasmosis) |

^{a}Approximate MIC based on Streptococcus pneumoniae, unless otherwise stated.

### EXAMPLES

### Example 1

### Treatment of bacterial Infection

A 70-year-old type II diabetic smoker with a diagnosis of acute exacerbation of chronic bronchitis is treated with cefuroxime acetil, administered transdermally to deliver a serum concentration of 16 μg/mL for 5 days, while avoiding the longer treatment period of 10 days required with oral twice a day.

A 3-year-old, penicillin-allergic boy with a diagnosis of acute, moderate-to-severe otitis media and nausea is treated with telithromycin, administered transdermally to deliver a serum concentration of 0.08-1.86 μg/mL for 5 days, while avoiding the unpleasant taste of the medication and concomitant difficulties in patient compliance and avoiding the necessity for intravenous infusion to achieve systemic administration and spare the gut.

A 53-year-old man with osteomyelitis of the hip, secondary to methicillin-resistant Stapholococcus aureus and C. difficile colitis after oral antibiotic treatment is treated with vancomycin, administered transdermally to deliver a serum concentration of 20 μg/mL for 7-28 days, while avoiding the necessity of intravenous delivery to spare further insult to the gut.

### Example 2

### Treatment of Viral Infection

A 25-year-old male is being treated for genital herpes zoster. To prevent flare-ups, this patient is administered transdermal acyclovir to deliver a serum concentration of 5-20 μg/mL and preferably 10 μg/mL for up to one year.

### Example 3

### Treatment of Mycobacterial Infection

A 30-year-old man diagnosed with active Mycobacterium tuberculosis infection is treated with rifampin, administered transdermally to deliver a serum concentration of 4-32 μg/mL and preferably 7 μg/mL for 52 weeks, providing a treatment with reduced likelihood of emergence of a resistant strain of the causative organism.

### Example 4

### Treatment of Fungal Infection

A 35-year-old woman, former intravenous drug abuser, HIV-positive, has been diagnosed with Candida albicans esophagitis. The patient is unable to take oral medication because of severe sore throat pain and intravenous administration is not feasible, and therefore is treated with transdermal Caspofungin, delivered to achieve a serum concentration of 75 μg/mL for two weeks.

### Example 5

### Treatment of Helmintic Infection

A 34-year-old woman is presumptively diagnosed with Trichinellosis, caused by Trichonella sprirosis after consumption of undercooked pork. Prior to confirmatory muscle biopsy, the patient is treated with transdermal Mebendazole, delivered to achieve a serum concentration of 10 μg/mL for three days.

### Example 6

### Treatment of Protozoan Infection

A 72-year-old woman with pernicious anemia who cares for ferile cats contracts Toxoplasmosis (infection with Toxoplasma gondii, confirmed by serology). To avoid development of folate deficiency to complicate the existing vitamin B12 anemia, the patient is treated with transdermal Pyrimethamine, delivered to achieve a serum concentration of at least 1 μg/mL and oral Sulfadiazine for about 4 weeks.

1. A method of improving the efficacy of and reducing the emergence of resistance in a time-dependent antimicrobial drug which comprises administering said time-dependent antimicrobial drug transdermally to achieve a serum concentration that remains above the MIC of said time-dependent antimicrobial drug.

2. A method of claim 1, wherein said time-dependent antimicrobial drug is selected from the group consisting of an antibacterial drug, an antimycobacterial drug, an antiviral drug, an antifungal drug, an antiprotozoan drug and an anthelmintic drug.

3. A method of claim 1, wherein said time-dependent antimicrobial drug is selected from the group consisting of penicillin, amoxicillin, oxacillin, dicloxoacilline, clavulinic acid, bicillin, ticarcillin, piperacillin, taxobactam, cephalexin, cefazolin, cephaclor, ceftibuten, cefuroxime, cefprozil, cefotaxime, ceftazidime, cefepime, cifdinir, ceftriaxone, cefditoren, cefpodoxime, aztreonam, ertapenem, cefoxitin, meropenem, imipenem, erythromycin, clarithromycin, azithromycin, telithromycin, clindamycin, daptomycin, cycloserine, quinupristin, dalfopristin, streptomycin, vancomycin and linezolid.

4. A method of treating a microbial infection in a patient with decreased risk of the emergence of resistance which comprises administering a time-dependent antimicrobial drug transdermally to said patient according to the method of claim 1.

5. A method of claim 4, wherein said time-dependent antimicrobial drug is selected from the group consisting of an antibacterial drug, an antimycobacterial drug, an antiviral drug, an antifungal drug and an anthelmintic drug.

6. A method of claim 4, wherein said time-dependent-antimicrobial drug is selected from the group consisting of penicillin,-amoxicillin, oxacillin, dicloxoacilline, clavulinic acid, bicillin, ticarcillin, piperacillin, taxobactam, cephalexin, cefazolin, cephaclor, ceftibuten, cefuroxime, cefprozil, cefotaxime, ceftazidime, cefepime, cifdinir, ceftriaxone, cefditoren, cefpodoxime, aztreonam, ertapenem, cefoxitin, meropenem, imipenem, erythromycin, clarithromycin, azithromycin, telithromycin, clindamycin, daptomycin, cycloserine, quinupristin, dalfopristin, streptomycin, vancomycin and linezolid.

7. A transdermal drug delivery device comprising a time-dependent antimicrobial drug and a pharmaceutically acceptable excipient.

8. A transdermal drug delivery device of claim 7, wherein said time-dependent antimicrobial drug is an antibiotic.

9. A transdermal drug delivery device of claim 7, wherein said time-dependent antimicrobial drug is a bacteriostatic drug.

10. A transdermal drug delivery device of claim 7, wherein said time-dependent antimicrobial drug is an antiviral drug.

11. A transdermal drug delivery device of claim 7, wherein said time-dependent antimicrobial drug is an antifungal drug.

12. A transdermal drug delivery device of claim 7, wherein said time-dependent antimicrobial drug is an anthelmintic drug.

13. A transdermal drug delivery device of claim 7, wherein said time-dependent antimicrobial drug in an antiprotozoan drug.

14. A transdermal drug delivery device of claim 7, wherein said time-dependent antimicrobial drug is selected from the group consisting of penicillin, amoxicillin, oxacillin, dicloxoacilline, clavulinic acid, bicillin, ticarcillin, piperacillin, taxobactam, cephalexin, cefazolin, cephaclor, ceftibuten, cefuroxime, cefprozil, cefotaxime, ceftazidime, cefepime, cifdinir, ceftriaxone, cefditoren, cefpodoxime, aztreonam, ertapenem, cefoxitin, meropenem, imipenem, erythromycin, clarithromycin, azithromycin, telithromycin, clindamycin, daptomycin, cycloserine, quinupristin, dalfopristin, streptomycin, vancomycin and linezolid.

15. A transdermal drug delivery device of claim 7, which further comprises a second time-dependent antimicrobial drug.

16. A method of treating a microbial infection in a patient which comprises administering systemically to said patient a transdermal drug delivery device of claim 7.

17. A method of systemically providing an antibacterial treatment to a patient while avoiding gastrointestinal side effects of said treatment, which comprises administering to said patient a transdermal drug delivery device of claim 7.

18. A method of systemically providing an antibacterial drug to a patient while lessening the likelihood of emergence of drug resistance to said drug, which comprises administering to said patient a transdermal drug delivery device of claim 7.
